(19) United States
(12) Patent Application Publication
(10) Pub. No.: US 2013/0009445 A1
(43) Pub. Date: Jan. 10, 2013

(54) TRACTION APPARATUS FOR USE WITH SEATS

(76) Inventor: Zachary COOPER, Ben Lomand, CA (US)

(21) Appl. No.: 13/219,601

(22) Filed: Aug. 26, 2011

Related U.S. Application Data
(60) Provisional application No. 61/377,435, filed on Aug. 26, 2010.

(51) Int. Cl. B60R 22/00; A61F 5/24

(52) U.S. Cl. 297/468; 128/95.1

(57) ABSTRACT

An apparatus is provided that includes a belt adapted for extending around the torso of a human user and coupling to a support so that at least a portion of the weight of the user is carried by the support through the belt.

### CROSS REFERENCE TO RELATED APPLICATION

This application claims priority to U.S. provisional patent application Ser. No: 61/377,435 filed Aug. 26, 2010, the entire content of which is incorporated herein by this reference.

### SCOPE OF THE INVENTION

The present invention relates to belts, and more particularly to belts for providing support to a human back.

### BACKGROUND

Belts have been provided for supporting the backs of users during lifting and certain other manual labor activities.

### BRIEF DESCRIPTION OF FIGURES

FIG. 1 is a front perspective view of a traction apparatus of the present invention in an open position.

FIG. 2 is a front perspective view of the traction apparatus of FIG. 1 in a closed position.

FIG. 3 is a front schematic view of a person using the traction apparatus of FIG. 1.

FIG. 4 is a side schematic view of a person using the traction apparatus of FIG. 1.

FIG. 5 is a rear view of a seat with the traction apparatus of FIG. 1 attached to the headrest posts of the seat.

FIG. 6 is a perspective view of another embodiment of the traction apparatus of the present invention for use with a seat mounted on a support surface.

FIG. 7 is an enlarged view of the bottom of the seat of FIG. 6 with a portion of the traction apparatus of FIG. 6.

FIG. 8 is a perspective view of the bottom of the seat of FIG. 6 with a portion of the traction apparatus of FIG. 6.

FIG. 9 is a perspective view of a portion of the traction apparatus of FIG. 1 being coupled to the headrest posts of a seat with a retention mechanism.

FIG. 10 is another embodiment of a retention mechanism suitable for use with the traction apparatus of the present invention.

FIG. 11 is a further embodiment of a retention mechanism suitable for use with the traction apparatus of the present invention.

FIG. 12 is another embodiment of a retention mechanism in use with a portion of the traction apparatus of FIG. 1 and the headrest posts of a seat.

FIG. 13 is a perspective view of a portion of another embodiment of the traction apparatus of the present invention coupled to the headrest posts of a seat.

FIG. 14 is a perspective view of a portion of a further embodiment of the traction apparatus of the present invention.

FIG. 15 is a perspective view of a portion of another embodiment of the traction apparatus of the present invention.

FIG. 16 is a perspective view of another embodiment of the traction apparatus of the present invention.

FIG. 17 is another perspective view of the traction apparatus of FIG. 16.

FIG. 18 is a plan exterior view of another embodiment of the traction apparatus of the present invention.

FIG. 19 is a plan interior view of the traction apparatus of FIG. 18.

### DESCRIPTION OF THE INVENTION

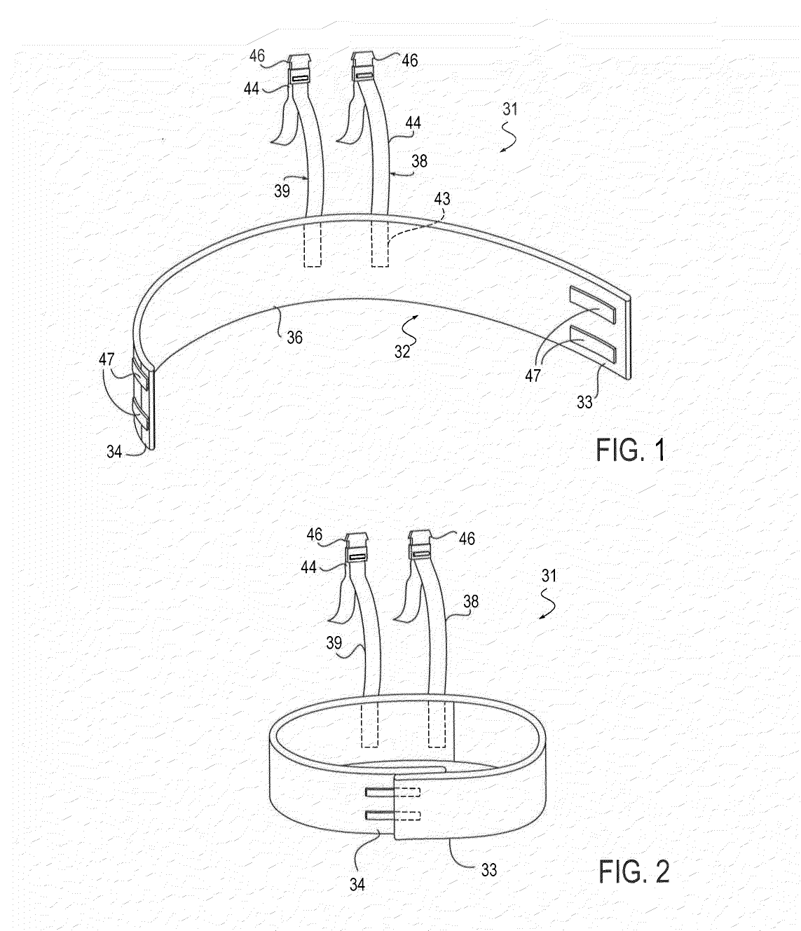

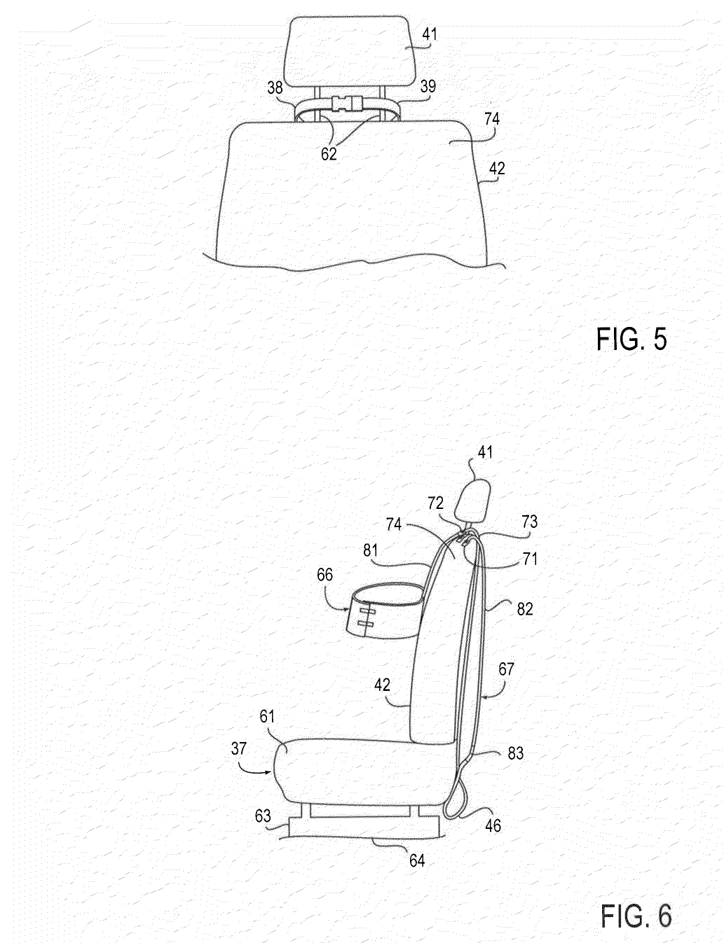

FIG. 1 is a frontal view of an opened traction device 31 or apparatus of the present invention. The device 31 includes a belt 32 sized for circumscribing the waist or torso of a human. The belt has first and second end portions 33,34 and a central portion 36 disposed between the end portions. At least one coupling element or strap is secured to the device for coupling the device 31 to a chair, seat or other support. Other means, besides the at least one strap, can be utilized for securing the belt to the chair, seat or other support 37. The belt 32 can be formed from any suitable flexible and bendable material, and can have a single layer of material or multiple layers of material. The one or more layers of the belt have a sufficient tensile strength so as to permit the belt to be fitted snugly around the waist or central portion of the user and support the weight of the user when so secured to the user. The at least one anchor strap can be secured to any portion of the belt, and in one embodiment is secured to the central portion 36 of the belt and extends substantially perpendicular to the belt. In the embodiment illustrated in FIG. 1, first and second spaced-apart straps 38,39 are secured to the central portion 36 of the belt. In one embodiment, the first and second anchor straps 38,39 are spaced apart so as to be generally centered on the spine of the user and extend up the back of the user. Although the anchor straps can extend parallel to each other, in one embodiment the straps are inclined outwardly as they extend upwardly from the belt so as to generally have the shape of the letter V; the outer ends of such outwardly inclined or V-shaped anchor straps can be attached to a headrest 41 atop the backrest 42 of a seat 37, and can particularly can at least partially wrap around such a headrest that is attached to the seat without posts. The belt is constructed from any suitable material capable of supporting the weight of a human body and preferably the weight of an adult human body. The anchor straps can be heavily sewn onto the outside or inside of the central portion of the belt so as to support the weight of a person wearing the belt. Each of the anchor straps 38,39 can range in size from 0.5 to 2.5 inches in width and can be made from any suitable material such as nylon, covered cord, durable rope, thread, belt, netting, rigid bracket, canvas, elastic, chain, rubber or plastic for anchoring the traction apparatus to a chair, seat or other support. Examples of such a chair, seat or other support 37 includes an automobile seat, a truck seat, a hauling vessel seat, a seat in heavy machinery, an office chair, a home chair, an airline seat, a bus seat, a train seat, a passenger seat and a seat or sitting device in any vehicle, vessel or transport structure or on any support surface. Covers for the straps can be fitted in to prevent wear and tear on the seats. Each of the anchor straps 38,39 can each have a first end portion 43 secured to the belt 32 by any suitable means such as by thread sewn through all layers of the belt. The second end portion 44 of each anchor strap that leads away from the belt 32, that is the free portion 44 of the anchor strap, can have adjustment hardware capable of determining the length of the strap between the belt and the end of the strap. In one embodiment, a detachable clip or other means or mechanism 46 for coupling the traction apparatus 31 to a chair, seat or other support 37 is provided on or in the vicinity of the free end of the anchor strap. Such a detachable clip (see FIGS. 5-15) on the anchor strap can clip into any suitable anchoring device so as to secure the traction apparatus 31 to a chair, seat or other support 37 such as an automobile, truck or other support structure. Such a detachable clip 46 can also connect to itself after looping around a portion of a fastener on an automobile, truck or other support structure.

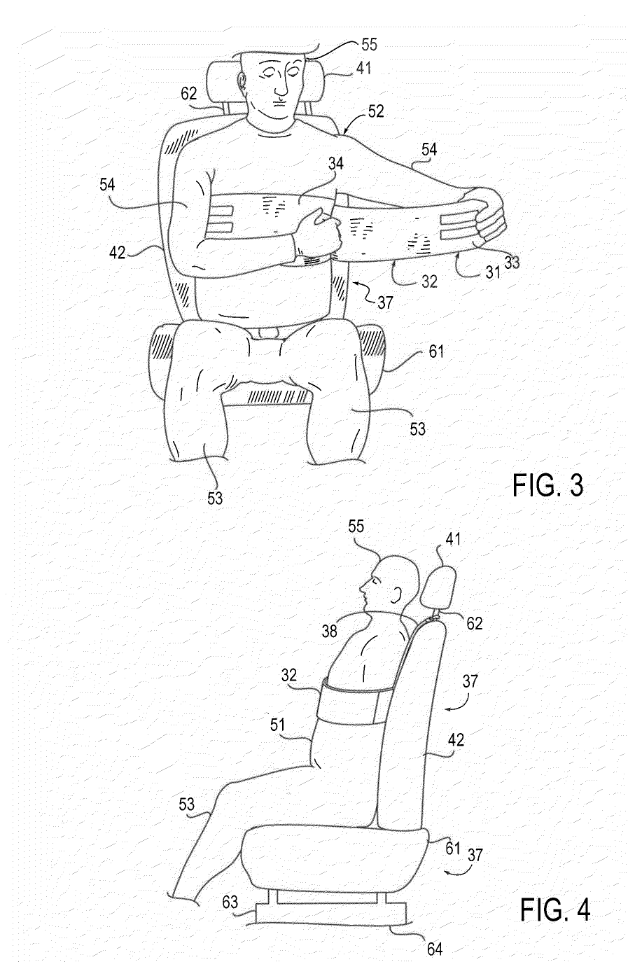

The first and second end portions 33,34 of the belt 32 can have any suitable cooperatively-configured fasteners 47 for securing together the first and second end portions of the belt around the waist or torso 51 of the human user 52. The human user can further include first and second legs 53, first and second arms 54 and a head 55, as shown in FIGS. 3-4. Suitable fabric hook-and-loop fasteners 47, for example as shown in FIGS. 1-2, include Velcro fasteners, which can be of any suitable size and shape capable of securing together the end portions 33,34 of the belt 32. The fasteners can be securely sewn to any portion of the belt, such as the interior of the belt, capable of keeping the belt tightly closed around the abdomen, waist or torso of an adult human body when wrapped around the human abdomen, waist or torso 51. The fabric fasteners can be in the form of one or more cooperatively-mating strips, as shown in FIGS. 1 and 2, or in the form of a sheet forming all or a portion of at least the first and second end portions of the belt. The belt can have a small strip of fabric that surrounds the edge of the belt in order to secure the two or more layers of fabric that the belt is comprised of. Velcro can also be sewn to an outer portion of the belt in order to further enhance the strength of the overall Velcro bond once the outer layer of the belt is wrapped over it.

In operation and use, the weight of the user 52 supported by the belt and traction apparatus 31 relieves pressure from the lower spine or skeleton of a person wearing the traction apparatus, for example a person seated in a chair 37 to which the straps 38,39 of the device are secured. The material used for the belts construction can be receptive to Velcro adhesive on its outer layer. The inside layer of material used for belt Construction can be soft or comfortable enough to accommodate contact with a human abdomen 51. The belt 32 has a length sufficient to accommodate the potential circumference of the human waist, torso or abdomen.

FIG. 3 illustrates a front view of a person 52 in the act of either opening or closing the belt 32 of the traction apparatus 31. The belt can be fitted with an additional band of material or handle that acts as a quick release. The person or user 52 is seated in a seat 37 having a bottom seat rest 61, a backrest 42 and a headrest 41 mounted atop the backrest by first and second headrest posts 63.

FIG. 4 is a side view of a person 52 wearing the traction belt 32 before attaching the coupling mechanisms 46 located at the ends of the straps 38,39 to the headrest posts 62 or other portions of the seat 37 or underlying seat support structure 63 which rigidly couples the seat 37 to a support surface 64 such as a floor or frame of an automobile, truck or plane. In method of operation, the first and second end portions 33,34 of the belt 32 are secured together around the waist or other portion of the torso 51 of the human user 52. The belt is then coupled to the seat, chair or other support 37, by for example coupling the free end portions 44 of the first and second straps 38,39 to the posts 62 securing the headrest 41 to the seat 37. The length of the straps is adjusted, either before or after securing the straps to the seat, chair or other support, so that the all or a portion of the weight of the user is supported by the straps of the traction apparatus 31.

FIG. 5 is a view from behind a seat 37 on which a traction device 31 is being utilized. In this illustrated embodiment, the free end portions 44 of straps 38,39 of the traction apparatus 31 loop around any headrest 41 or other raised portion of the seat or chair 37. In the illustrated embodiment of FIG. 5, the first and second straps 38,39 loop to the rear of the first and second posts 62 securing the headrest 41 to the seat back 42 and are secured together by the cooperatively-mating coupling mechanisms 46 carried by the ends of the straps. Many types of fasteners and fastening techniques can be used in order to secure the device's straps 38,39 behind the seat or chair. The straps can be widened in order not to cut into a seat 37 that has no headrest 41. Any height adjustment of the traction apparatus 31 relative to the seat, chair or other support 37 can be achieved by loosening or tightening the anchor straps 38,39 with a conventional tension bracket or noose (not shown in FIG. 5) which can be an appendage of the coupling mechanism or suitable bracket 46 the traction apparatus 31 employs to secure the ends 44 of the first and second straps 38,39 together.

FIG. 6 illustrates another embodiment of the traction apparatus of the present invention. Traction apparatus or device 66 therein is substantially similar to traction apparatus 31 and like reference numerals have been used to describe like components of traction devices 66 and 31. First and second anchor straps 67,68 of the traction device 66, substantially similar to anchor straps 38,39, are anchored at the bottom and rear of the car seat 37 where the support structure 63 of the seat 37 attaches to the frame or body 64 of the car or other vehicle. A suitable bracket 71, as illustrated, may be needed at or near the top of the seat 37 in order to prevent the first and second straps 67,68 from falling over the shoulders of the seat. Conventional tightening appendages 72 can be added to the anchor straps 67,68, for example at the shoulder area or where the intermediate portion 73 of the straps drape over the back of the seat 37. The belt portion 32 of the device 31 can separate from the seat or vehicle frame at the shoulder area 74 of the seat or near the top of the seat by providing that anchoring straps 67,68 are segmented. The segmented straps can, for example, have a first portion 81, illustrated in FIG. 6, that extends from the belt to or near the shoulder area 74 of the seat or other area and a second portion 82 that removably secures to the first portion 81 by any suitable means and extends from the first portion to the chair, seat or other support 37, the support structure 63 for the seat or the support surface or frame 64 to which the seat support structure 63 is rigidly secured. In FIG. 6, the free end portion 83 of each second portion 82 of the straps is coupled to the seat support structure 63 by coupling mechanisms 46 or any other suitable means. In such embodiment, the user can easily access the point of securement of the first portion 31 to the second portion 82, for example located near the shoulder area of the seat, to detach the first portion 81 and thus the belt 32 from the second portion 82 of the segmented straps.

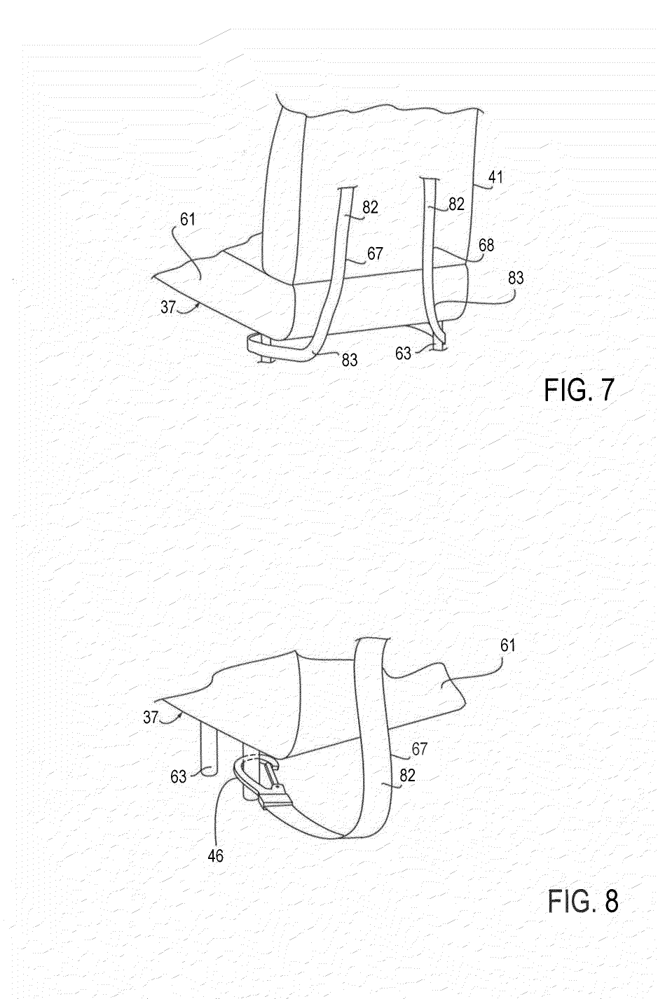

FIG. 7 is a view of the embodiment of the traction apparatus 66 of FIG. 6 in which the first and second segmented or unitary anchor straps 67,68 secured to the belt 32 of the device extend over the top of the backrest 42 of a suitable seat 37, such as an automobile seat, and down the rear of the seat. The free end portion 83 of the second portion 82 of each of the first and second straps 67,68 secure to the seats posts or anchor of the seat support structure 63 or to the frame 64 of the vehicle by any suitable means, such as for example by looping around such seat posts, anchor or frame and securing together. It is appreciated that the one or more anchor strap of the traction apparatus 66 can secure directly to the chair, seat or other support 37 by for example coupling to such chair, seat or other support 37.

FIG. 8 shows a coupling mechanism 46 at the end of an anchor strap 67,68 of traction device 66 that includes a clip that may be used to anchor the free end portion 83 of the strap to its self after being passed through the undercarriage or seat support structure 63 of an automobile seat 37 or to the frame 64 to which the seat support structure 63 is rigidly coupled. This hook, clip or other coupling mechanism 46 can also be used to clip directly to any part of the seat 37 or even ceiling or other upper portion of the cab of the auto or vehicle if such an elevated hanging anchor exists or if the belt 32 is being used for example in a big rig, large hauling vessel or similar transporter vehicle.

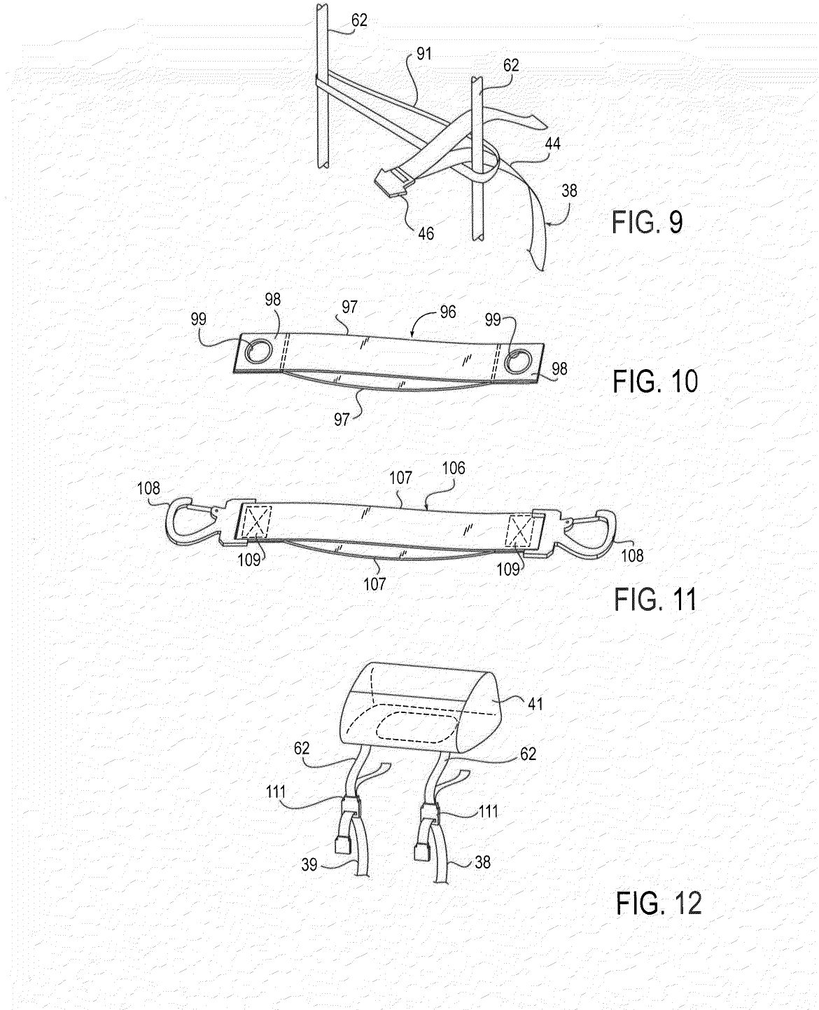

FIG. 9 is a view of an anchor strap 38,39 of the traction apparatus or device 31 illustrated in FIG. 1 passing through an elastic, rubber, cotton, or soft securing material or device 91 in order to keep the anchor strap of the device 31 from sliding off of the shoulder or top 74 of an auto or other seat 37. It is appreciated that other forms of brackets or mechanisms 91 can be utilized for retaining the anchor strap in proper position relative to the seat, chair or other support. In the embodiment of FIG. 9, such retention mechanism 91 is in the form of a loop that extends around and between the posts 62 for securing the headrest to the seat 37. The anchor strap 38 extends between the two parallel portions of the loop 91 and between the headrest posts 62.

FIG. 10 is another embodiment of a suitable retention mechanism for retaining the anchor straps in proper position relative to the chair, seat or other support. Such retention mechanism 96 includes first and second parallel strips 97 of flexible material, for example made from nylon or another durable material, that are secured together at each of their respective first and second ends 98 by any suitable means. A hole 99 is formed in each end 99 of the retention mechanism, for example through the material of the first and second strips 97 at the ends 98 thereof. The first and second posts 62 of a removable auto headrest 41 can be passed through the holes 99 in the material in order to create a secure place for the anchor straps of the traction device of the present invention to rest without risk of falling off of the shoulder 74 of the seat 37.

FIG. 11 is another embodiment of a suitable retention mechanism 106 that is similar to the retention mechanism 96 of FIG. 10. The first and second short strips or straps 107 of retention mechanism 96 are secured together and a clip, hook or similar mechanism 108 is provided on each end 109 of the retention mechanism 106 for enabling it to be secured to an auto headrest 41 without removing the headrest. Such retention mechanism 106 helps to secure the anchor straps of the traction apparatus or device of the present invention as they pass over the back of the auto seat 37.

FIG. 12 shows a retention mechanism that includes hooks 111 attached to the anchor straps 38,39 of traction apparatus 31 or any other traction apparatus of the present invention for clipping to the headrest posts 62 as the straps 38,39 pass over the auto seat 37 towards a suitable anchor to which the straps 38,39 are coupled or secured. The hooks 111 can slide freely on the straps 38,39, making them automatically height adjusted. When the straps 38,39 are so secured to the headrest 41 and to any other desired anchor of the vehicle, the straps 38,39 would be less likely to fall off the seat back 42 or otherwise move around the seat 37.

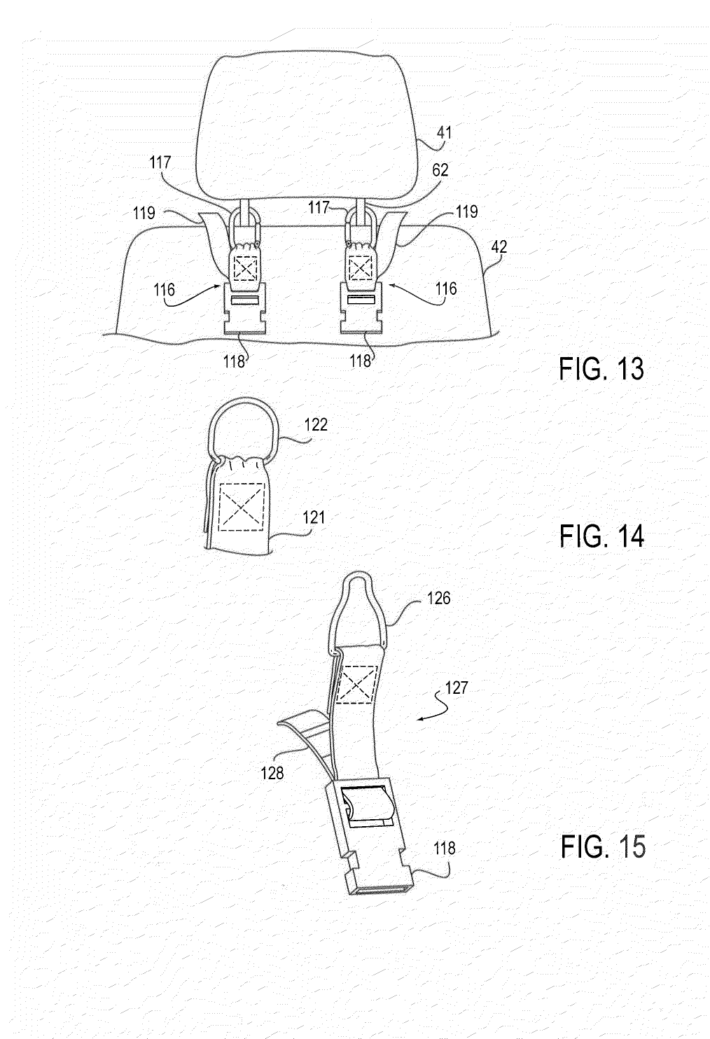

FIG. 13 illustrates first and second fastening mechanisms 116, each of which includes a clip or carabineer 117, that clip to a headrest post 62, undercarriage or desired anchor for the traction apparatus of the present invention. In the embodiment of FIG. 13, the fasteners 118 below the carabineers of the fastening mechanisms 116 clip to the ends of the anchor straps of the traction apparatus of the present invention so as to permit the traction apparatus to be easily attached to and removed from the seat, chair or other support 37. Conventional strap length adjustment mechanisms 119 can be included in the fastening mechanisms 116 for adjusting the length of the fastening mechanisms and thus desirably positioning the belt 32 of the traction apparatus relative to the chair, seat or support 37 and permitting such positioning to be easily repeated when the traction apparatus is detached and then reattached to the seat, chair or other support.

FIG. 14 is an embodiment of an anchor strap of the traction apparatus of the present invention, for example anchor strap 121 that is similar to anchor strap 38, that is sewn directly onto a metal or durable material ring 122 that can be secured to an auto headrest 41 or headrest post 62 or similar support by removing the auto headrest 41 and passing the post 62 of the headrest through the ring 122. The end of the anchor strap 121 can be secured to the ring 122 in any suitable manner, such as by looping through the ring and then being sewn to a portion of the anchor strap adjacent such end of the anchor strap. Such a ring or similar fastener 122 secured to the end of each of the first and second anchor straps of the traction apparatus of the present invention would permit such anchor straps to be secured to the first and second headrests posts 62 of the auto seat 37 and thus anchor the traction apparatus to the auto seat 37.

FIG. 15 is a view of a carabineer or bell shaped loop 126, similar to ring 122, that can be opened and that can be used to clip an anchor strap of the traction apparatus of the present invention to a post 62 of a headrest 41 or anchoring area of an automobile. Such clip or loop 126 can also be utilized in a fastening mechanism 127, for example similar to the fastening mechanism 116 shown in FIG. 13. The clip 126 can be sewn or otherwise securely attached to the nylon or other material of the strap 128 of the fastening mechanism 127 in order to anchor the strap 128 to the chair, seat or other support 37. At least two of such clips 126 may be necessary in order to so anchor the belt 31 of the traction apparatus and create lift for the wearer, such as human user 51, of the belt.

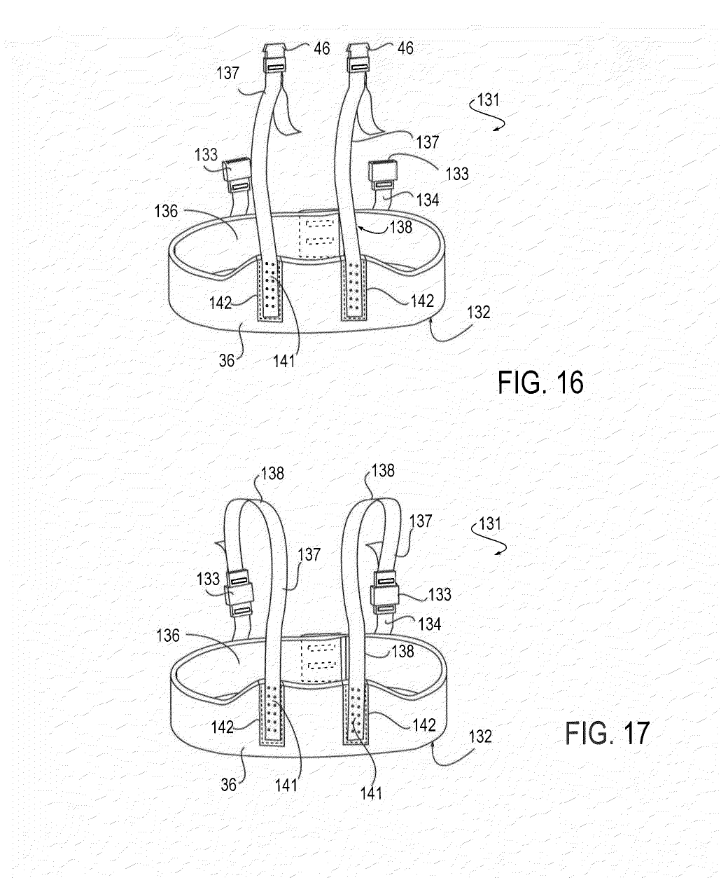

FIG. 16 is a rear view of another embodiment of the traction apparatus of the present invention. Such traction apparatus or device 131, similar to apparatus or device 31 and like reference numerals used to describe common components of devices 31 and 131, includes a belt 132, substantially similar to belt 32 and illustrated detached from the seat, chair or other support 37. Such embodiment includes two clips or fasteners 133 on the front 136 of the belt 132. Each of such clips or fasteners 133 can be secured to a strap 134 that is sewn or attached by any suitable means such as fabric hook-and-loop fasteners (not shown) to the front 136 of the belt 132, and can cooperate with so as to secure to a coupling mechanism 46 at the free end portion 137 of the respective anchor strap 138, which can be similar to anchor straps 38,39, so as to permit the anchor straps 137 to resemble pants suspenders. The second end portion 141 of each anchor strap 138 can be secured to the central portion 36 of the belt 132 by any suitable means such as being sown or by hook-and-loop fasteners 142. When the user 52 of the traction apparatus 131 wants to separate the device 131 from the seat, chair or other support 37 and walk around or move freely, the first and second clips 133 on the front 136 of the belt 132 receive the coupling mechanisms 46 of the first and second anchor straps 138 after the anchor straps have been extended or draped over the shoulders of the user and thus permit ease of movement by the user wearing the traction apparatus 131 and lack of drag from the traction apparatus.

FIG. 17 is a rear view of the belt of the traction apparatus 131 of FIG. 16 with the belt 132 in the closed position and the first and second anchor straps 138 coupled to the first and second clips 133 so as to resemble suspenders.

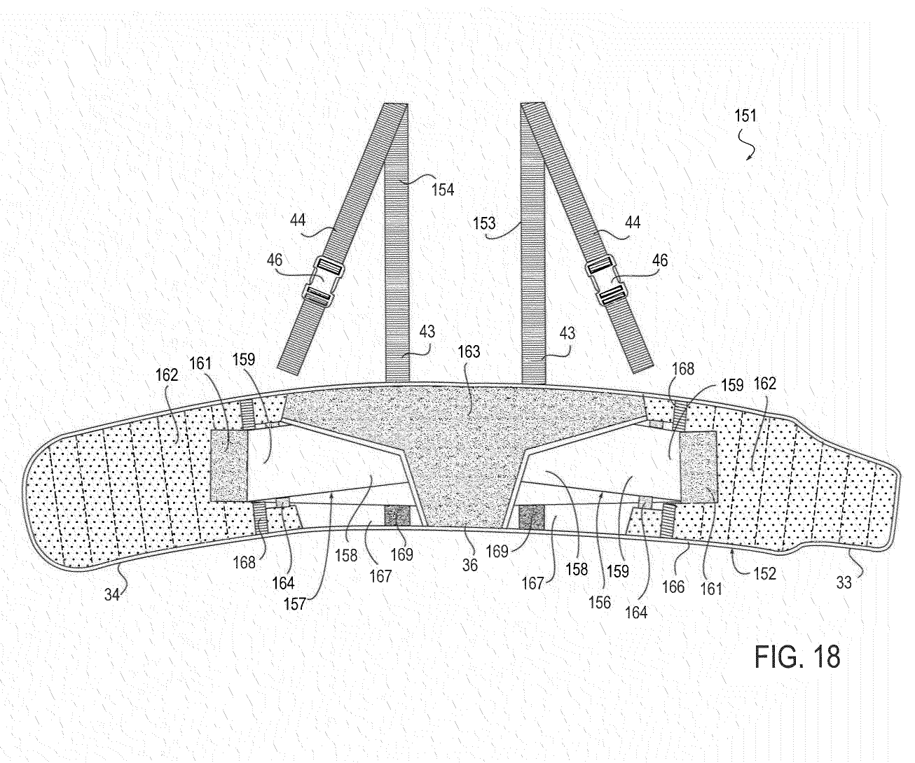

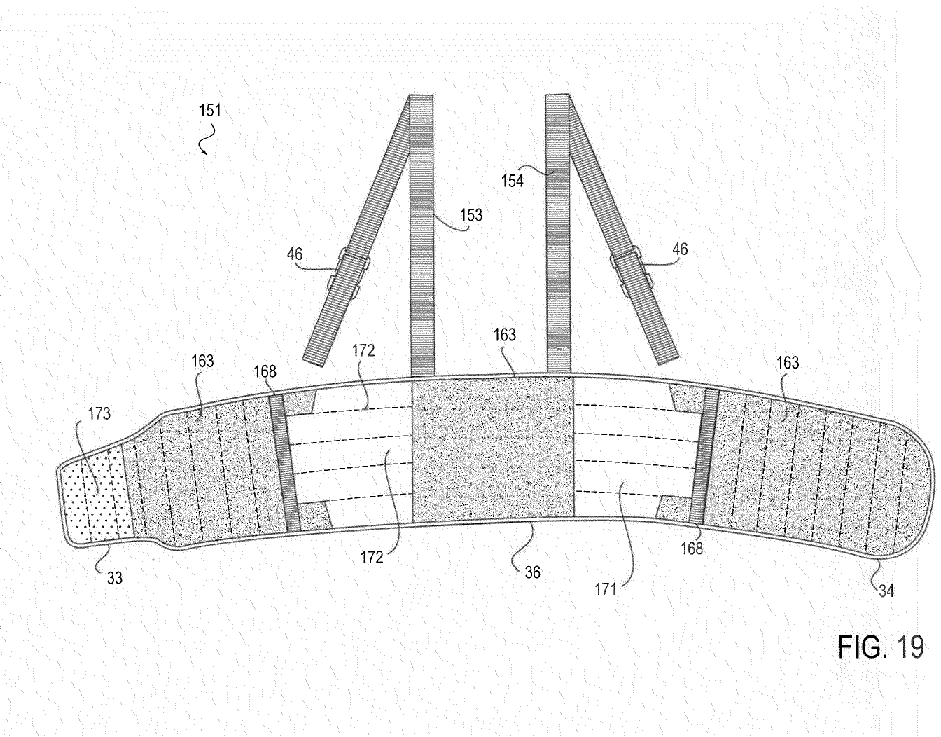

Another embodiment of the traction apparatus of the present invention is illustrated in FIGS. 18-19. Traction apparatus or device 131 therein is substantially similar to apparatus 31 and like reference numerals have been used to describe like components of apparatus 31 and 151. The traction device 151 includes a belt 152 substantially similar to belt 32 and first and second adjustable tension or anchor straps 153,154 substantially similar to first and second anchor straps 38,39. Suitable coupling mechanisms such as quick release adjustable buckles 46 are provided on the free end portion 44 of each strap 153,154. Belt 152 includes first and second adjustment tension or elastic straps 156,157 that each have a first end portion 158 secured to the central portion 36 of the belt 152 and an opposite second or free end portion 159 extending along the belt away from the central portion 36 and having an adjustment strap hook tab 161 at the end thereof. Each of the straps 156,157 can be formed from a loop of a suitable elastic material, with each end of the loop secured to the central portion 36 of the belt and the hook tab 161 secured to the center portion of the loop. Each of the elastic straps 156,157 can be stretched to an appropriated tightness after the belt 152 is attached to the user 52 and the hook tab 161 thereof fastened to the loop fastening panel 162 provided on the outside of the belt 152 at each of first and second end portions 34,34 of the belt 152. The hook and loop fastening system 161,162 allows the adjustment tension straps 156,157 to be completely removed from the device or adjusted within the device 151. Belt 152 can include first and second secondary adjustment straps 164 that extend around at least the inner portion of each loop forming an adjustment strap 156,157 so as to hold the adjustment straps into a secure position when all of such straps 156,157,164 are engaged. At least a portion of the belt 152 can be formed from a breathable mesh liner 163.

The elasticized secondary adjustment straps 156,157 allow the wearer 52 to tighten or loosen the belt 152 by securing the hook tabs 161 of the straps 156,157 to the respective loop fastening panel 162, an act that is possible from the front of the device 151 while leaving the belt 152 secured on the wearer. The breathable mesh liner 163 acts as a cage for the adjustment straps 156,157 securing such straps to the device 151 incase such straps are torn open in the front unintentionally. The breathable mesh liner cage 163 keeps the straps 156,157 from dangling. The adjustment straps 156,157 each can include a hook tab 161 for securing to the loop fastening panel 162. The stabilizing strap 164 for the elastic portion of each secondary adjustment strap 156,157 provides stability for the secondary adjustment straps and helps keep them in place behind the breathable mesh liner 163.

The elasticized bindings 166 allow the device to wrap around the user without cutting off circulation. Note the arch of the device 151 its self as it is contoured in order to comfortably wrap around the wearer underneath the arms; the arch of the device also leads the device to slip downward while being worn thus keeping it in the desired area of the body where it can be of the most use.

The expansion netting panels 167 allow air to flow through the device in order to further comfort the wearer and alleviate sweat. There are two pliable yet rigid stabilizing couplers 168 sewn into the device 151 that further the stability of the device; the stabilizing couplers 168 make the device easier to secure to the wearer as they hold the shape of the device to a comfortable form before being coupled. The device 151 can additionally include a pocket (not shown) within the breathable mesh liner 163 in the rear center 36 of the device, for example for an ice pack. The device can additionally include a further fastening system (not shown) in the tension strap adjustment panel 169 for the adjustable tension or arch straps 153,154 which can be in the form of brackets, snaps, buttons, clips, pins, ties, laces or any combination of fasteners with hook and loop fabric.

Traction device 151 can use various high tech fabrics sewn throughout the device in order to anchor the devices tension and adjustment straps 153,154,156,157. The stabilizing couplers 168 are rigid yet flexible and designed to bend to the contour of the wearer. The breathable expansion netting panels 171 and reinforced stitching 172, each shown on the interior of the belt 152 in FIG. 19, and linear placement of fabrics appropriately coordinated with opposing lines in order to create an aesthetic appeal. The breathable mesh liner 163 is a thicker and more durable fabric placed appropriately in order to provide anchoring for the hook and loop fastening system 161, 162 of the secondary anchoring straps 156,157 as well as the primary hook fastening panel 173, on the first and second end portions 33,34 of the device 151. The breathable mesh liner 163 and anchor stitching 174 in the center of the device 151, for securing each of the straps 156,157 to the central portion 36 of the belt 152, provide stability for the adjustment straps 156,157 as well as hook and loop adhesion for the adjustable tension straps allowing them to detach or remain secure and supportive. The multiple layers of porous fabric placed throughout the device 151 give it a supportive girth while remaining ventilated and breathable.

Some additional features of the traction apparatus of the present invention can include:

The traction apparatus can be used with a car, a truck and a home chair, as an attachment for the interior door of a home or a recreational vehicle, and at a doctors office;

Many attachments such as a lipstick holder, a pen holder, a cell phone holder, a gun holder, and a wallet holder can be attached to the belt of the traction apparatus by any suitable means including fabric hook-and-loop fasteners;

Accessories can be provided, and the traction apparatus can facilitate a massage of the back of the user. For example, a vibrating massage mechanism, a heat coil or wire or any other back treating device can be attached to the belt and/or the anchor straps in the vicinity of the portion of the user's body to be treated;

The traction apparatus can reduce compression in the skeleton and spine of the user, which compression can impair organ function;

Multiple colors, sizes, width and lengths of the traction apparatus can be provided to fit population variety;

The traction apparatus can be provided with a feminine shape, with a sized chest protrusion. Such a feminine shape can include a possible curvature in the belt where the anchor straps connect to the belt;

The belt of the traction apparatus can be made from various grades of materials, with parameters for material selection including budget and comfort;

Clips or other coupling mechanisms can be provided at the ends of the anchor straps for attaching the anchor straps to a headrest, around the lower frame of chair, the ceiling or the cab of a vehicle;

The traction apparatus can include adjustable straps with multiple fastening configurations, including the ability to loop through the bottom of a seat, clip to a seat or a chair, anchor to a vehicle frame and/or clip to a headrest, and can include a loop of nylon or other material for use with a headrest to couple the anchor straps to the headrest and an attachment for an interior door of a home;

The anchor straps of the traction apparatus can be adjusted within arm reach while driving, and can include a clip for the straps to detach behind the shoulders of the user;

An embodiment of the traction apparatus can be provided with anchor straps long enough to become suspenders and can include one or more clip receptors on the front of belt for coupling to the fasteners or coupling mechanisms on the ends of the anchor straps so as to couple the ends of the anchor straps to the front of the belt;

The traction apparatus can include Velcro or other cloth hook-and-clip fasteners with an optional extra strap or straps that circumscribe the apparatus; and

The traction apparatus can be machine washable and have a quick release handle, and can include pockets, a gun holster or cell phone holder appropriately coated with Velcro or coupling mechanisms for attaching to the belt of the apparatus.

It is appreciated that numerous other embodiments of the traction apparatus can be provided that accomplish the intended purpose of the traction apparatus and utilize some or all of the features disclosed in the foregoing embodiments of the traction apparatus.

I claim:

1. An apparatus for use with a support by a human having a torso, comprising a belt adapted for extending around the torso of the human, the belt having first and second end portions and a fastener attached to at least one of the first and second end portions for securing the first and second end portions together, and at least one coupling element having a first end secured to the belt and a second end provided with a coupling mechanism adapted for securing to the support whereby when the belt is secured around the torso of the user and the coupling element is secured to the support at least a portion of the weight of the user is carried by the support through the coupling element.

2. The apparatus of claim 1, wherein the belt has a central portion between the first and second end portions and the first end of the at least one coupling element is secured to the central portion of the belt.

3. The apparatus of claim 2, wherein the at least one coupling element includes first and second coupling elements having first end portions secured to the central portion of the belt.

4. The apparatus claim 1, wherein the at least one coupling element is a strap.

5. A method for supporting the back of a human in a seat of a vehicle, comprising securing a belt around the torso of the human and attaching the belt to one of the seat and the vehicle so that at least a portion of the weight of the human is carried by the belt and the weight carried by the lower back of the human is reduced.

6. The method of claim 5, wherein a coupling element is secured to the belt and has a free end and wherein the attaching step includes attaching the free end of the coupling element to one of the seat and the vehicle.

7. The method of claim 5, wherein the seat includes a headrest and wherein the attaching step includes attaching the belt to the headrest of the seat.

8. The method of claim 5, wherein the seat includes a rear and a base and wherein the attaching step includes attaching the belt to the base of the seat at the rear of the seat.

9. The method of claim 8, wherein a strap is secured to the belt and has a free end and wherein the attaching step includes attaching the free end of the strap to base of the seat at the rear of the seat.

10. The method of claim 9, wherein the seat includes a backrest having a top portion and wherein the strap has an intermediate portion and further comprising coupling the intermediate portion of the strap to the top portion of the seat.
